(19) United States
(12) Patent Application Publication
(10) Pub. No.: US 2008/0294022 A1
(43) Pub. Date: Nov. 27, 2008

(54) Birthing Medical Monitor

(75) Inventors: Yehuda Sharf, Tel-Aviv (IL); Moshe Betzalel, Modiin (IL); Yuri Megel, Haifa (IL)

Correspondence Address: MARTIN D. MOYNIHAN d/b/a PRTSI, INC., P.O. BOX 16446, ARLINGTON, VA, 22215, US

(73) Assignee: BARNEV LTD., Netanya (IL)

(21) Appl. No.: 10/599,693

(22) PCT Filed: Nov. 29, 2004
(86) PCT No.: PCT/IL2004/001092

§ 371 Date: Jul. 29, 2008

Related U.S. Application Data
(60) Provisional application No. 60/560,291, filed on Apr. 7, 2004.

(51) Int. Cl. A61B 5/00

(52) U.S. Cl. 600/304

(57) ABSTRACT

A birth monitoring system (100), comprising a monitor (120) including at least one sensor operative to measure a physiological parameter associated with labor; a posture sensor (130) which generates a signal indicative of posture of a mother (110); and circuitry which generates an output signal dependent on both a measurement of said sensor and a posture measurement of said posture sensor (130).

### RELATED APPLICATIONS

The present application claims the benefit under 35 USC 119(e) of U.S. provisional application Ser. No. 60/560,291, filed Apr. 7, 2004, the disclosure of which is incorporated herein by reference.

### FIELD OF THE INVENTION

The present invention relates to the field of medical monitors, for example for monitoring birth.

### BACKGROUND OF THE INVENTION

Monitors are commonly used in medicine for tracking physiological parameters of patients. In some cases, the use of a monitor allows data to be collected continuously from a patient, during daily activities or when there are no caregivers around. In some cases, a monitor is used to reduce the workload of care providers.

In PCT publication WO 02/098271 from Dec. 12, 2002, the disclosure of which is incorporated herein by reference, there is described a birth monitoring system, which, inter alia, monitors the dilation of the cervix during birth.

### SUMMARY OF THE INVENTION

An aspect of an embodiment of the invention, relates to a medical monitor that optionally modifies its output, or an output signal thereof, responsive to measurements corresponding to a posture of a monitored patient. For example the output of a medical monitor for the measurement of the progress of birthing is modified depending on whether the mother is lying on her back or on her side, or walking. In an exemplary embodiment of the invention, the modification takes into account a difference in meaning or in measurement of the physiological parameter, dependent on the posture of the patient. In some embodiments of the invention, the modification takes into account a change in configuration of the medical monitor. As used herein the term posture is used in its regular meaning of the relative positions of various body parts to the whole and relative to gravity.

In some embodiments of the invention, the functionality of or a response to the medical monitor is modified based on the posture measurements, for example an alarm is not initiated. In some embodiments of the invention, values displayed by the monitor are adjusted based on the measurements.

In an exemplary embodiment of the invention, the posture is determined using an inclination sensor. Optionally, the inclination sensor is positioned in its own separate encasement on the patient. Optionally, the separate encasement is positioned next to the medical monitor. Alternatively, the inclination sensor is encased in the medical monitor's encasement. In some embodiments of the invention, the inclination sensor is positioned inside the patient, for example on the cervix. In some embodiments, multiple inclination sensors are used, to provide more posture information.

In an exemplary embodiment of the invention, the posture sensor is used for a birth monitoring system and is used to take into account changes in maternal geometry caused by changes in position of the mother, for example, changes in relative placement of internal sensors with inclination of the mother, changes in relative positions of external sensors or relative placement of internal sensors relative to external sensors.

In an exemplary embodiment of the invention, the posture sensor is calibrated to have an orientation matching one or more axes of the mother, for example the longitude axis and the lateral axis. In an exemplary embodiment of the invention, the posture sensor is provided in a housing which has a marking which can be aligned with one or both axes. Alternatively or additionally, the housing is oriented towards a landmark, for example, the navel.

In an exemplary embodiment of the invention, the posture sensor is separable from the housing and is calibrated, during manufacture, to have a known alignment relative to the housing. In one example, the inclination sensor fits in the housing only in a particular orientation and the calibration is relative to that orientation.

In an exemplary embodiment of the invention, the attachment site of the sensor is desirably fixed, since it is used as a reference to an internal marker/sensor. In some embodiments, a plurality of internal sensors whose relative position is meaningful, are used. Optionally, the inclination sensor is calibrated/fixed relative to rigid parts of the mounting system. Optionally, change in inclination is used to detect that the reference function of the attached sensor is compromised or changed.

An aspect of some embodiments of the invention relates to a calibration method in which a rapid change in a geometrical value is assumed to be an artifact caused by change in posture, rather than a real change. Optionally, the change in posture is measured using a posture sensor. Optionally, the calibration is effected by generating a correction value which corrects a post-posture change value to be the same as a pre-posture change value. In an exemplary embodiment of the invention, a posture sensor is used to determine when a patient has returned to a previous position and then the correction value may be zeroed.

An aspect of some embodiments of the invention relates to tracking, monitoring and/or causing an effect of positional or other maternal bodily changes on physiological parameters relating to labor and birth. In an exemplary embodiment of the invention, in a birth monitoring system, it is determined if fetal vital signs changes as a result of position change, e.g., to see if bradycardia improved. In another example, the effect of movement of the mother's legs on advancing of the head is tracked. In another example, the effect of maternal breathing on fetal head motion is monitored. In an exemplary embodiment of the invention, a birth monitor suggests positional or other changes in the mother, for example, breathing or turning over. Optionally, the monitor checks to see if the instructions were followed and/or their effect.

An aspect of some embodiments of the invention relates to a mounting method for mounting of sensors on a pregnant women's abdomen, including an elastic adhesive pad with a window for an ultrasonic sensor. In an exemplary embodiment of the invention, the window is an aperture. In an exemplary embodiment of the invention, the window is an ultrasonically transparent material. In an exemplary embodiment of the invention, the elastic adhesive pad is able to maintain adhesion under conditions of contraction and expansion of the abdomen, during labor.

An aspect of some embodiments of the invention relates to a two part mounting system for attaching a sensor to skin surface of a body, including a ring adhesive section with at least one snap mounting attachment thereon and a sensor holding section adapted to removably snap-mount on said ring adhesive section. In an exemplary embodiment of the invention, when mounted, the sensor is pressed into a skin surface. In an exemplary embodiment of the invention, the ring section comprises a fitting that mounts utilizing an adhesive ring to provide the snap mounting attachment. Optionally, the snap mounting is a twist-snap mounting.

An aspect of some embodiments of the invention relates to a method of coating an ultrasonic sensor with acoustic coupling gel, in which a cover of an adhesive part of a sensor mounting system is removed, together with excess gel after the gel is placed on the sensor. In an exemplary embodiment of the invention, gel may be applied by detaching the sensor from the mounting system after the sensor is mounted on the body and placing gel in a thus-exposed aperture. Optionally, one or more apertures are provided adjacent the sensor for outlet of excess gel.

An aspect of some embodiments of the invention relates to detecting maternal physiological parameters, such as contractions and/or breathing using acceleration or inclination sensors. Optionally, a same sensor as used for detecting a physiological parameter is used to detect maternal posture. Optionally, such maternal physiological parameters are tracked to determine their relationship with fetal parameters. Optionally, the sensor is used for generating an alert when labor progresses sufficiently to justify usage of internal (e.g., cervical) sensors. Optionally, the external sensor is mounted on a halter device.

There is thus provided in accordance with an exemplary embodiment of the invention, a birth monitoring system, comprising:

a monitor including at least one sensor operative to measure a physiological parameter associated with labor;

a posture sensor which generates a signal indicative of posture of a mother;
circuitry which generates an output signal dependent on both a measurement of said sensor and a posture measurement of said posture sensor. Optionally, said output signal comprises a signal selectively suppressed responsive to said measured posture. Alternatively or additionally, said circuitry is operative to modify said output signal responsive to said posture signal. Alternatively or additionally, said circuitry is operative to generate a signal indicative of a change in said physiological parameter responsive to a change in said posture.

In an exemplary embodiment of the invention, said physiological parameter comprises a geometry of a cervix. Alternatively or additionally, said physiological parameter comprises a fetal head position relative to a birth canal of a mother. Alternatively or additionally, said physiological parameter comprises a fetal physiological parameter. Optionally, said physiological parameter comprises a fetal heart rate.

In an exemplary embodiment of the invention, said physiological parameter comprises a maternal physiological parameter.

In an exemplary embodiment of the invention, said physiological parameter is changed by a change in posture.

In an exemplary embodiment of the invention, a measurement by said sensor is changed by a change in posture. Optionally, said sensor comprises at least one internal probe and at least one external probe serving as a reference thereto.

In an exemplary embodiment of the invention, said output signal is generated if a posture change is not maintained.

In an exemplary embodiment of the invention, the system comprises a memory which stores a correspondence between posture and a physiological parameter.

In an exemplary embodiment of the invention, said circuitry extracts at least one maternal physiological parameter from said posture sensor. Optionally, said maternal parameter comprises breathing.

In an exemplary embodiment of the invention, said posture sensor comprises an acceleration sensor.

In an exemplary embodiment of the invention, said posture sensor is calibrated to be aligned to one or more maternal body axes.

In an exemplary embodiment of the invention, said output signal comprises a recommendation to a caregiver regarding posture.

In an exemplary embodiment of the invention, said posture sensor is housed together with at least a part of said at least one sensor.

There is also provided in accordance with an exemplary embodiment of the invention, a method of generating an output signal indicative of a medical monitoring of a patient in labor, comprising:

measuring at least one postural parameter of the patient;

measuring at least one physiological parameter associated with labor; and

generating a signal responsive to both of said measurements. Optionally, the method comprises discarding a physiological measurement responsive to a posture measurement. Alternatively or additionally, said generating comprises correcting a physiological measurement based on said posture measurement. Alternatively or additionally, said generating comprises tracking a change in at least one physiological parameter responsive to said posture measurement. Alternatively or additionally, said generating comprises generating a physiological measurement from said posture measurement.

In an exemplary embodiment of the invention, the method comprises monitoring an effect of posture change on said physiological parameter.

In an exemplary embodiment of the invention, the method comprises monitoring a compliance of a patient with a posture change.

In an exemplary embodiment of the invention, the method comprises determining a side on which the patient is lying.

In an exemplary embodiment of the invention, said postural parameter and said physiological parameter are both acquired using a same sensor. Optionally, the method comprises determining a change in posture based on a measured change in said at least one physiological parameter.

In an exemplary embodiment of the invention, correcting comprises applying a correction value to said physiological parameter. Optionally, the method comprises updating said correction value when a posture change is detected. Optionally, updating comprises updating assuming that said physiological parameter does not change over a period of time of the occurrence of said posture change.

There is also provided in accordance with an exemplary embodiment of the invention, a mounting assembly, comprising:

an elastic adhesive ring adapted to adhere to a human skin;

a selectively locking mount attached to said ring; and

a housing adapted to receive a sensor and selectively lockable to said mount, and configured to provide mechanical contact of at least one part thereof to a skin to which the ring adheres. Optionally, said locking is a snap mounting. Alternatively or additionally, said mount interlocks with at least one aperture in said ring. Alternatively or additionally, said sensor comprises an ultrasonic transducer adapted to ultrasonically communicate through a center of said ring and wherein said mechanical contact is suitable for ultrasonic transmission therethrough. Alternatively or additionally, said sensor comprises an inclination sensor.

In an exemplary embodiment of the invention, the assembly comprises circuitry for RF communication.

In an exemplary embodiment of the invention, the assembly comprises a power source in said housing.

In an exemplary embodiment of the invention, the assembly comprises circuitry for digitizing and processing of ultrasound signals.

There is also provided in accordance with an exemplary embodiment of the invention, a method of applying ultrasound coupling material to an active element designed for gel-coupled contact with skin, comprising:

mounting the element in an adhesive mount;

applying a coupling material suitable for ultrasonic coupling to the element; and

stripping a covering from said mount, thereby exposing an adhesive layer of said mount and removing excess gel-like material.

### BRIEF DESCRIPTION OF THE DRAWINGS

Particular non-limiting exemplary embodiments of the invention will be described with reference to the following description of embodiments in conjunction with the figures, wherein identical structures, elements or parts which appear in more than one figure are generally labeled with a same or similar number in all the figures in which they appear, in which:

FIG. 1 is a schematic illustration of implementing a birth monitor system with the use of an inclination sensor, in accordance with an exemplary embodiment of the invention;

FIG. 2 is a flow diagram of a method of modifying a medical monitor or its output, in accordance with an exemplary embodiment of the invention;

FIG. 3 is a schematic block diagram of a monitoring system coupled to an inclination sensor in accordance with an exemplary embodiment of the invention;

FIG. 4 is a flowchart of a method of using inclination information to track the effect of posture changes, in accordance with an exemplary embodiment of the invention;

FIG. 5 shows a monitoring assembly including a plurality of sensing or transmission units, for attachment to a controller, in accordance with an exemplary embodiment of the invention;

FIGS. 6A-6D show various views of a sticker section and of an attachment element, in accordance with an exemplary embodiment of the invention;

FIG. 7 shows an interlocked sticker section and attachment element of FIGS. 6A-6D;

FIGS. 8A-8E show a housing section for interlocking with the sticker section of FIG. 7, in accordance with an exemplary embodiment of the invention;

FIG. 9 shows an assembled mounting unit utilizing the parts shown FIGS. 5-8 in accordance with an exemplary embodiment of the invention; and

FIG. 10 is a flowchart of a method of gel application in accordance with an exemplary embodiment of the invention.

### DETAILED DESCRIPTION OF EXEMPLARY EMBODIMENTS

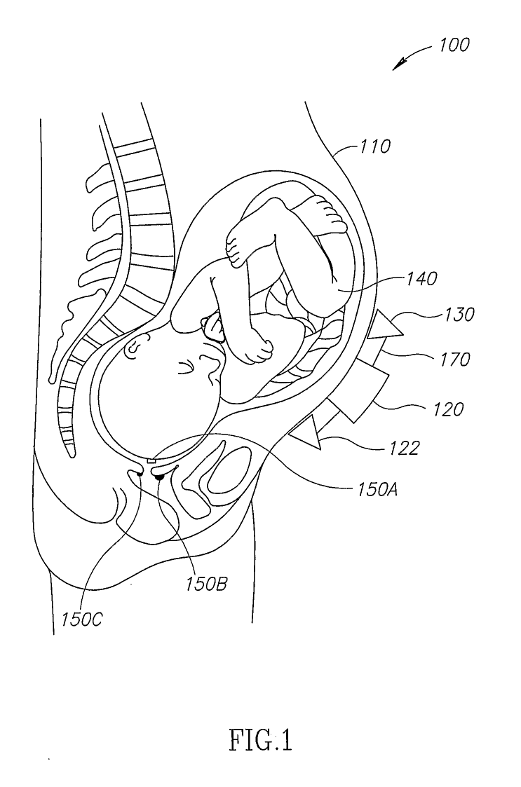

FIG. 1 is schematic illustration of an implementation of a birth monitor system 100 with an inclination sensor 130 being used for posture determination, in accordance with an exemplary embodiment of the invention.

In an exemplary embodiment of the invention, a female patient 110 with a fetus 140 is connected to a monitor 120, for example as described in WO 02/098271 or in 60/560,291, filed Apr. 7, 2004, the disclosures of which are incorporated herein by reference. Monitor 120 optionally monitors vital signs of patient 110 and/or fetus 140, for example heart beat. Additionally, one or more probes 150 (for example 150A, 150B and 150C) may be placed on the cervix of patient 110 and optionally on the head of fetus 140 for used in tracking the geometry of the cervix and/or fetal head.

In an exemplary embodiment of the invention, probes 150 act as position sensors whose relative positions can be determined. As described in the above WO publication, the positions of probes 150 can be determined using an external (to the body) transmitter and/or receiver, for example an ultrasonic and/or RF receiver/transmitter 122. Time of flight and/or other methods are used to determine the distance between probes 150 and receiver/transmitter 122, so that the relative geometry of probes 150 can be determined. The inventors have realized that for a same birth progress situation, changing the posture of the mother can change the relative positions of probes 150 and 122, and possibly affect the correctness of the measurement of the maternal and fetal geometry and/or its interpretation.

In an exemplary embodiment of the invention, the posture of patient 110 is estimated using an inclination sensor 130. Optionally, measurements by sensor 130 enable monitor 120 to take into account the position and/or change in position of patient 110.

In an exemplary embodiment of the invention, inclination sensor 130 is positioned on the body of the patient, for example on the patient's abdomen above the pubic bone near monitor 120, as shown in FIG. 1. In some embodiments of the invention, the location of inclination sensor 130 is dependent on the type of monitor 120. For example, for some monitors inclination sensor 130 is located on a central part of the patient, for example the trunk of a patient, for example on soft tissue such as the abdomen, which may deform, or on hard tissue such as the Anterior Superior Iliac Spine (e.g., the pelvic bone), which will not deform (but overlying soft tissue may deform). Alternatively, inclination sensor 130 is positioned on the same part of the body as the monitor or the part of the body being monitored by monitor 120 (e.g. the cervix), in order to report on the inclination of the part to which the monitor is connected. For example, the abdomen is expected to deform as the patient changes posture. Further alternatively, inclination sensor 130 may be located elsewhere, for example attached to the monitor itself or for example on an arm or a leg of patient 110 while sleeping or in a coma. Optionally, multiple inclination sensors are used, for example, on one or both legs and on the back and/or abdomen, thus possibly providing further postural information, such as angle between legs, angle between legs and trunk and/or deformation of the trunk. Optionally, the information provided includes a level of activity of the patient.

In an exemplary embodiment of the invention, one or more transponders (e.g., reference 122) are provided for communication with probes 150, and an inclination probe is provided packaged with one or more of the transponders.

In some embodiments of the invention inclination sensor 130 and monitor 120 are connected by a cable 170. Alternatively, inclination sensor 130 and monitor 120 communicate with a wireless connection, for example RF.

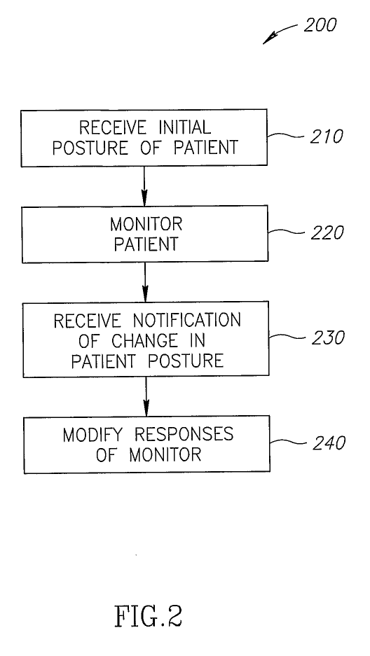

FIG. 2 is a flow diagram 200 of a method of modifying the functionality of monitor 120 based on the posture of a patient, in accordance with an exemplary embodiment of the invention. Initially, when starting the use of monitor 120, monitor 120 receives (210) from posture/inclination sensor 130 an initial notification of the inclination of patient 110. Monitor 120 starts monitoring (220) the patient taking into account the initial posture. Optionally, the initial inclination is entered manually. In some embodiments of the invention, inclination sensor 130 gives notification to monitor 120 when a change occurs, for example the inclination of patient 110 changes by more than 30° or 45° relative to the normal. Alternatively, inclination sensor 130 notifies monitor 120 of the inclination of patient 110 periodically, for example once a minute. In some embodiments of the invention, inclination sensor 130 notifies when a significant change occurs and/or at set time intervals. Optionally, sensor 130 provides substantially continuous or periodic measurements and monitor 120 determines changes.

In an exemplary embodiment of the invention, the patient is intentionally caused to change posture so that the effect of posture on the physiological and/or posture measurements can be assessed and/or monitor 120 calibrated. Optionally, such calibration is repeated during the treatment (e.g., labor), for example once an hour. Additional optional details regarding calibration are described below.

When monitor 120 receives (230) notification of the change in the inclination of patient 110, it checks if the inclination change is of an angle that will affect the meaning of the monitored measurements. In one example, a measurement from a patient lying on her side in a lateral supine position, will have a different meaning than same value determined in a measurement from a patient lying on her back. If monitor 120 determines that the change should effect the results (e.g., based on a calibration) it modifies (240) the responses of monitor 120. In one example, a measured value is modified, for example, increased, decreased and/or multiplied by a factor. Non-linear corrections may be applied as well. Optionally, the correction is applied by warping a geometry reconstructed from the measurements. Optionally, such changes in the measurements take into account a progress of the patient. For example, the modification in a head position calculation may depend not only on the patient's posture but also on the fetal head position (e.g., last correct value, if not too old). Other body postures changes may be detected as well, for example walking (which may cause artifacts and may be practiced as a walking epidural) and changes to/from standing. It should be noted that the maternal abdomen is expected to move around when the patient moves, due to its distension and its lack of rigid support. In an exemplary embodiment of the invention, the side the patient lies on (e.g., left or right) and/or the angle is tracked. In an exemplary embodiment of the invention, the body posture information is used to assess a correlation thereof with fetal state.

In an exemplary embodiment of the invention, the birth monitor operates with an accuracy of about 1 mm. In an exemplary embodiment of the invention, such accuracy can be maintained if an inclination angle measurement is about 1 degree. Alternatively or additionally, a lower accuracy may be used, for example, 5 degrees, which is optionally sufficient to generate alerts regarding maternal and/or fetal state.

In an exemplary embodiment of the invention a table or function (e.g., provided by calibration) is used to determine a value for updating the output of monitor 120. Optionally, only certain posture positions are legal for providing measurements, with intermediate positions ignored. Alternatively, an interpolation may be performed. Alternatively or additionally, a lesser weight may be associated with measurements at certain inclinations, for example inclinations where noise levels are higher or where distinguishability between different situations is reduced.

In an exemplary embodiment of the invention, the following ongoing calibration process is used to calibrate the measurement. In this process it is noted that real changes in maternal or fetal geometry occur slowly, while artifacts due to posture change are relatively fast. In an exemplary embodiment of the invention, a last measured geometry value is used as a true value. A value measured after posture change is assumed to represent a situation which is continuous with the old value. An offset is calculated which corrects the value measured after posture change to be the same as the value before posture change. Optionally, this offset is stored as part of a correction table for later use. Optionally, the offset is reset to zero when an initial posture is reached and the offset values table is optionally corrected to compensate for any difference between zero and the offset value. In an exemplary embodiment of the invention, the offset value is used to correct a fetal head position. Alternatively or additionally, the offset value is used to correct a cervical dilation value. In an exemplary embodiment of the invention, it is assumed that a reach change in value takes at least 10 seconds or 1 minute, while a posture related change takes 10 seconds or less, often as little as 1-2 seconds. The exact values may depend, for example on the patient or on a calibration procedure. Optionally, the typical times for a particular patient are measured automatically.

Optionally, the offset is updated when inclination change passes a certain value, for example, 1 degree.

In an exemplary embodiment of the invention, the offset type (e.g., additive, multiplicative) depends on the method of determination of the maternal or fetal geometry. For example, while a zero order offset is described, a higher order offset may be used as well. In one example, a non-linear correction is used in the input of a triangulation function (e.g., for locating the probes).

In an exemplary embodiment of the invention, monitor 120 optionally, cancels or initiates an alarm in response to a measured inclination. In one example, when a same measurement value in one posture is a critical state and in a second posture the same value is a normal value, a suitable alarm may be generated, which also clarifies if the state is critical or not.

In some embodiments of the invention, inclination sensor 130 gives an indication if the patient is in the correct inclination to be monitored, for example while using a monitor 120 that requires patient 110 to be in a specific posture. Optionally, if patient 110 is in the wrong posture, monitor 120 will give an indication for example by displaying an error message on a display of monitor 120.

In some embodiments of the invention, a patient 110 to which a monitor 120 is attached may be situated in different postures. Optionally, monitor 120 is calibrated based on the posture in which it is attached, or based on a main posture for use. In an exemplary embodiment of the invention, an indication is given to the user to calibrate the monitor upon initialization. In some embodiments of the invention, monitor 120 calibrates itself by accepting an initialization signal from inclination sensor 130 when it first starts or on request from monitor 120. Optionally, the initialization signal gives an absolute value, for example the offset angle from a normal to the earth. Further measurements can give measurements relative to the initial signal.

Optionally, alternatively or additionally to using a separate inclination sensor, properties of changes in geometry values are used to indicate changes in posture. For example, as noted above, real changes in maternal and fetal geometry are expected to be slower than changes in such factors caused by changes in the patient's position. Alternatively or additionally, postural changes are expected to have a similar or known type of effect on all measured positions of internal sensors. Alternatively or additionally, real changes, unlike postural changes are expected to be synchronized to contractions (e.g., as measured by TOCO or IUP). In an exemplary embodiment of the invention, such detection of possible changes in posture are used to determine when ongoing calibration (described above) should be applied, or to detect if an inclination sensor is properly located and/or operating.

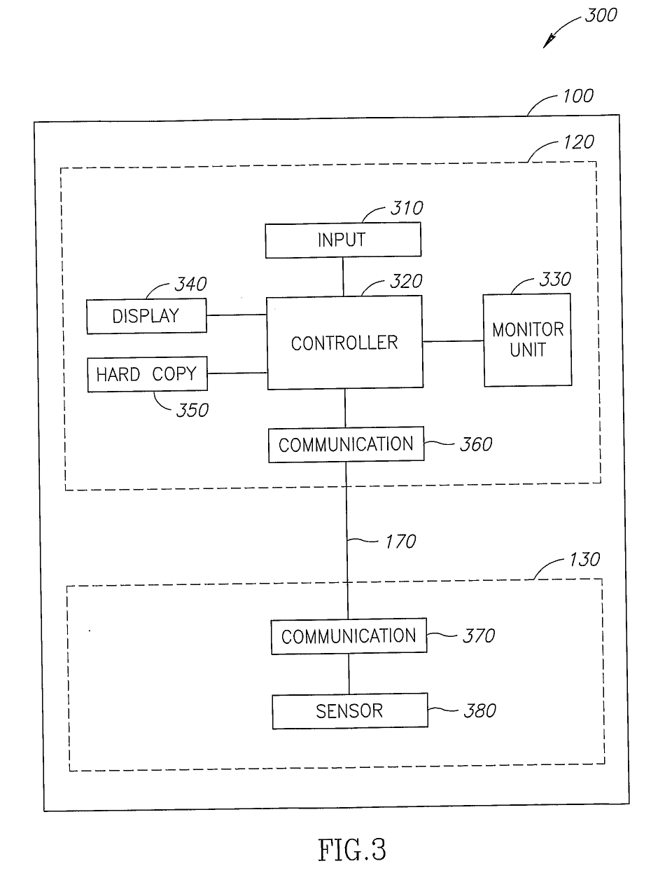

FIG. 3 is a schematic block diagram 300 of system 100 comprising an inclination sensor 130 and a monitor 120, according to an exemplary embodiment of the invention. As shown in FIG. 3, monitor 120 comprises a monitor unit 330, which monitors physiological parameters of a patient. In the above description, monitor unit 330 was exemplified as a birth monitor unit, however, other types of posture-dependent medical monitors can be supported, for example, a monitor being used to monitor a sleeping patient or a patient in a coma. In some embodiments of the invention, system 100 is used for a somnambulist.

In some embodiments of the invention, system 100 measures the rate of change of the inclination. For some medical situations and/or sensors, the rate of change is important in determining the effect measured. For example, if acceleration sensors are used to detect contractions, accelerations caused by walking may need to be taken into account.

In an exemplary embodiment of the invention, measured physiological parameters from monitor unit 330 are transferred to a controller 320, which analyzes the measurements and converts them to a displayable format and/or gives an alarm when needed. The measurements are optionally displayed on a display 340 or output to a hardcopy device 350, for example a plotter as commonly used in an ECG. Display 340 optionally includes alarm devices such as bells or flashing lights or transmissions to call for assistance.

In some embodiments of the invention, monitor 120 comprises an input device 310 such as a keypad or buttons, which can control parameters of monitor 120 or be used to reset and/or calibrate the device.

In an exemplary embodiment of the invention, monitor 120 comprises a communication interface 360 to communicate with inclination sensor 130. In some embodiments of the invention, communication interface 360 is connected via cable 170 as mentioned above, to a communication interface 370 of inclination sensor 130. Alternatively, other methods are used to connect between communication interface 360 and communication interface 370, for example wireless transmission. In some embodiments of the invention, monitor 120 and inclination sensor 130 are two separate devices that update each other as needed, for example monitor 120 notifies inclination sensor 130 to start or stop sensing and inclination sensor 130 transmits the sensed data to monitor 120. Alternatively, monitor 120 and inclination sensor 130 are constructed as a single device, for example in the same encasement, optionally sharing the same controller 320 without a communication interface between them.

Generally, the angular amount that the orientation of an object varies relative to a vertical plane is referred to as the tilt. The angular variation of an object relative to a horizontal plane is referred to as the inclination. Unless otherwise indicated, these three terms (inclination, tilt and posture), are used interchangeably with regard to sensors.

While the term inclination sensor has been used, there are a variety of sensors which are incorporated under that term and may be used as posture sensors. In an exemplary embodiment of the invention, inclination sensor 130 comprises a sensor 380, which incorporates a tiltmeter or an inclinometer using methods known in the art, for example as described in chapter 15 of “The Measurement, Instrumentation and Sensors Handbook (ISBN: 0-8493-8347-1)”, dated 1999, the disclosure of which is incorporated herein by reference. Exemplary tilt meters include acceleration meters, beams with stain gauges and gravity direction detectors.

In some embodiments of the invention, an accuracy of 1°-5° or 1°-10° for a selected inclination sensor 130 is sufficient for updating monitor 120. However inclination sensors with an accuracy of fractions of a degree, such as a tenth of a degree or less are also available. In some embodiments of the invention, for some monitors 120, sensor 380 is only required to differentiate between an upright patient and a supine patient for example to determine if the patient is sleep walking. In some embodiments of the invention, more than one inclination sensor is attached to a patient in order to follow body movements and/or relative position of various parts of the body, for example to determine if a patient is sitting or standing or which side of the body the patient is lying on. In some embodiments, the sensor is used to detect changes in posture, rather than absolute values.

In some embodiments of the invention, sensor 380 senses only in one direction and needs to be situated in a specific direction on patient 110. Alternatively, sensor 380 may sense inclination in two or three dimensions and may be situated more freely on the patient.

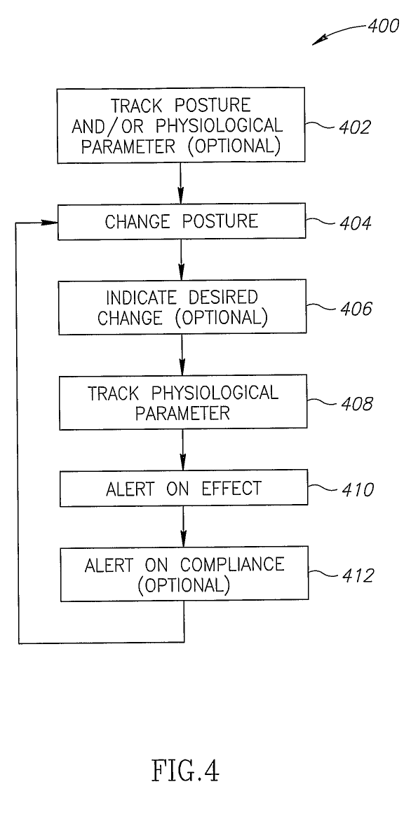

While the above has generally assumed that a posture change is a result of movement by the patient for independent reasons (e.g., pain discomfort, convenience), it should be noted that in some situations, such as labor, posture changes are mandated by the treating physician, for example for alleviating fetal distress. A common “treatment” for fetal distress is to have patient 110 roll on her side. FIG. 4 is a flowchart 400 showing how an inclination sensor is used to assist in such treatment and follow-up thereof, in accordance with an exemplary embodiment of the invention.

Optionally, system 100 continuously keeps track of the inclination and of one or more maternal and/or fetal physiological parameters, such as fetal heartbeat, maternal heart rate, fetal O₂ level, maternal blood pressure, intrauterine pressure, TOCO, cervical dilatation, and/or fetal head station/position. Optionally, a record is kept showing a relationship between the inclination and one or more physiological parameters. Optionally, a plurality of posture changes are performed so that their effect on the physiological parameter can be assessed. Optionally, the posture changes are used to assist in ongoing calibration of system 100. Optionally, one or more of the following postural parameters are tracked: standing, sitting or lying down, what side (or back or stomach) is lying down on, position of thighs relative to body, angle between legs, relative position of thighs, position of arms and/or angle of head. As noted, some of these postural parameters may use more than one orientation or inclination sensors. Alternatively or additionally, a postural parameter may include one or more of: a change, a rate of change, an absolute value (e.g., of a postural change or body position), a degree of synchronization between changes in positions of different body parts and/or a synchronization between a posture change and a maternal physiological parameter, such as breathing or breathing rate.

At 404, a decision is made that the patient should change her posture, for example, in an attempt to alleviate fetal distress. Optionally, the record made at 402 is examined to estimate if a change in posture will help and/or what change to perform.

At 406, the desired posture change is optionally indicated by a care provider to system 100. Optionally, a desired change in the physiological parameter is also indicated. Optionally, the caregiver indicates both in a code or other short indication. For example, a single indication may mean “turn patient on left side to help with bradycardia”. In an exemplary embodiment of the invention, system 100 tracks the physiological parameters to see if the posture change helped. Alternatively or additionally, system 100 tracks the patient posture to see if the posture change was executed and/or maintained.

In an exemplary embodiment of the invention, system 100 suggests the posture change (for example lying on side, spreading legs and/or deeper or shallower or other type of breathing) in response to a detected physiological parameter value. Alternatively, system 100 suggests that posture change is not suitable, for example if a previous posture change had no effect on a certain physiological parameter or had a negative effect.

At 408, the physiological parameter is tracked for changes. Such tracking may be manual. Optionally however, system 100 or an associated birth monitoring system keeps track of the changes and their effects.

At 410, an alert is optionally generated responsive to whether a desired (or undesired) change in the physiological parameter occurred (or not).

At 412, an alert is optionally generated responsive to whether patient 110 complied and/or is continuing to comply with the posture change instruction.

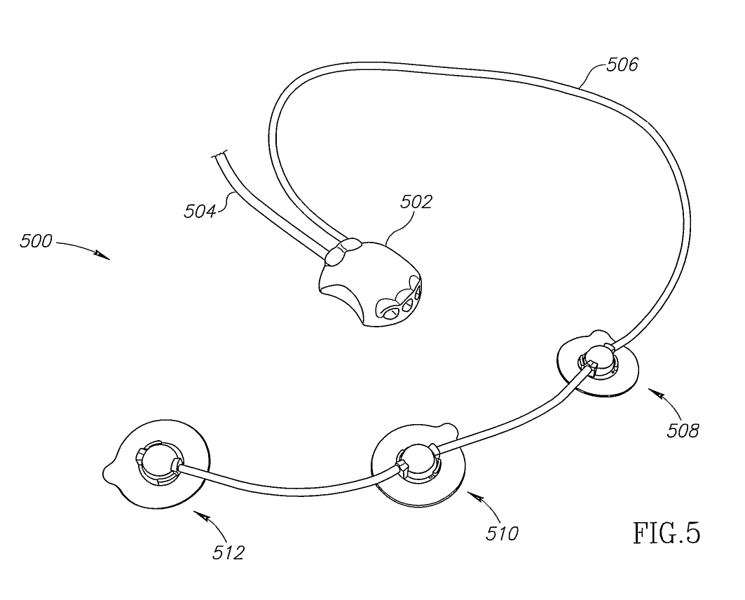

FIG. 5 shows a birthing monitor 500 (excluding a controller which is not shown) including a plurality of sensing or transmission units, in accordance with an exemplary embodiment of the invention. In the exemplary embodiment shown, an amplifier unit and a connector box 502 for the internal sensors located on the thigh (not shown) is attached by a cable 506 to a plurality of ultrasonic transmitting units 508, 510 and 512.

In an exemplary embodiment of the invention, an inclination sensor is provided in one or more of units 508-512. Alternatively or additionally, an inclination sensor is provided in box 502. A cable 504 optionally provides a power and data connection to a processing unit (not shown). A potential advantage of using multiple inclination sensors is in the detection of leg movements relative to the abdomen.

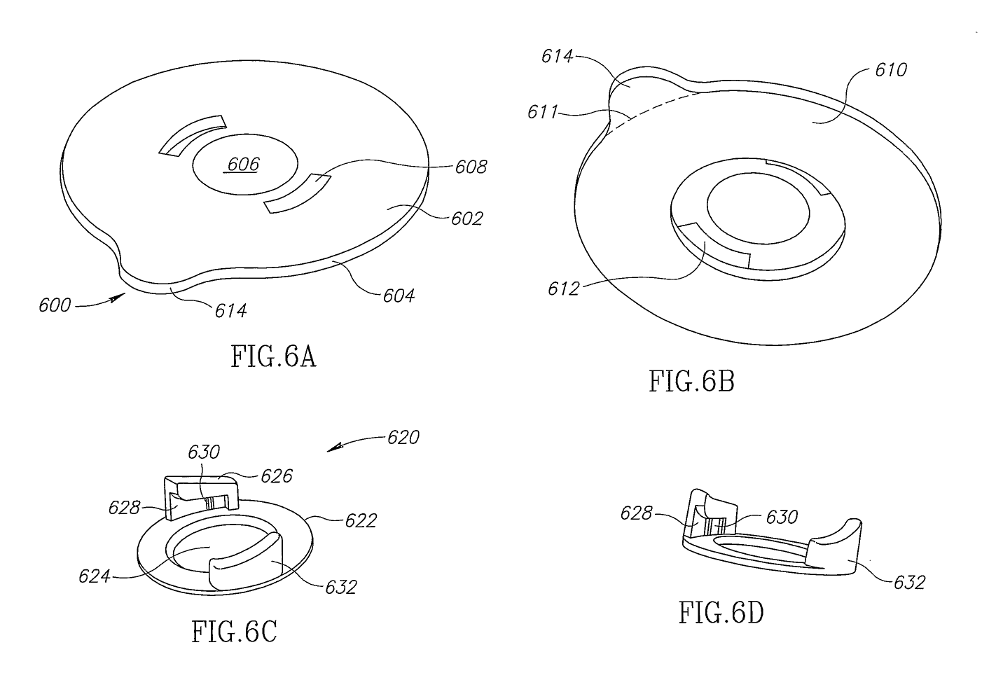

Each of units 508-512 optionally has a same design, of a sticker section with a snap attachment (or other attachment method) and a housing that mounts on the snap attachment. FIG. 6A-6D show different views of a sticker section 600 and of a snap attachment element 620, in accordance with an exemplary embodiment of the invention. Optionally, section 600 and element 620 are attached in factory, for example using adhesive or ultrasonic bonding.

FIGS. 6A and 6B are perspective top bottom views of sticker section 600. In an exemplary embodiment of the invention, three layers are provided; an upper, thin, flexible film 602, on which a layer of foam adhesive 604 is provided, and a layer of removable liner 610 for selectively exposing the adhesive foam. In an exemplary embodiment of the invention, foam 604 is 1-1.6 mm in thickness and film 602 is 0.05 mm in thickness. In an exemplary embodiment of the invention, film 602 is flexible so that it can stretch with changes in the abdomen geometry during labor. The adhesive is optionally flexible and designed to maintain adhesion to skin for a period of several hours, such as 24 hours. A tongue 614 is optionally provided for easy grasping of liner 610. Optionally, a scoring 611 is provided in liner 610, so that when the liner is removed tongue 614 remains covered with paper which can be used to lift off section 600. Alternatively, tongue 614 is not coated with adhesive.

An aperture 606 is optionally defined in the layers for accommodating a ultrasonic transmitter, so that the transmitter can directly contact the skin of patient 110 through the aperture. Alternatively, only layers/materials that do not significantly degrade the transmitted signal are allowed in the aperture. If the mounting system described herein is used for sensors, layers that do not interfere with the sensed signal are used.

FIGS. 6C-6D are perspective views from above and side respectively of snap attachment element 620. In an exemplary embodiment of the invention, element 620 includes projections that fit in designated apertures formed in section 600, so that the two parts interlock. In an exemplary embodiment of the invention, element 620 is in the shape of a ring 622 with an aperture 624 that matches aperture 606 and optionally has a thickness to fit within an area 612 that is clear of foam in the bottom of section 600. A first projection 626 and a second projection 632 project from ring 622 and are adapted to fit through designated apertures 608 in section 600. An interlock mechanism between section 600 and element 620 is optionally provided, for example by projections 626 and 632 including a narrow waist.

In an exemplary embodiment of the invention, each of projections 626 and 632 is designed to interlock with a housing cover described below. In particular, projection 626 is shown as including a groove 628 defined therein for receiving a wing of the housing cover. A cantilevered cut-out beam 630 is optionally provided for snap interlocking with a matching notch in the wing. Alternative interlocking mechanisms can be used as well.

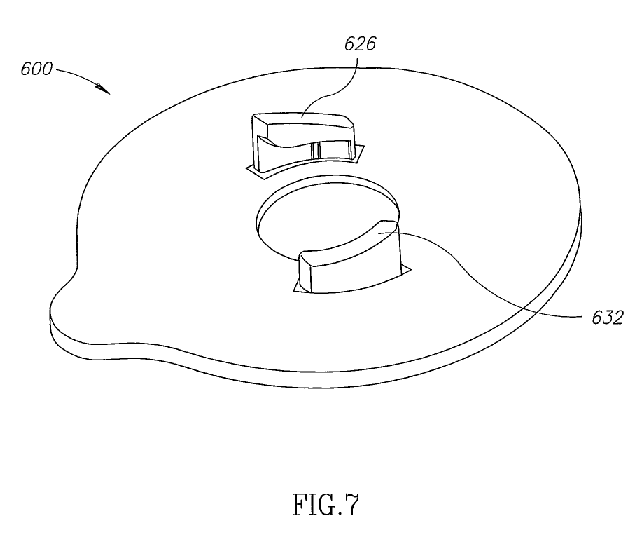

FIG. 7 shows an interlocked sticker section 600 and attachment element 622 (showing projections 626 and 632).

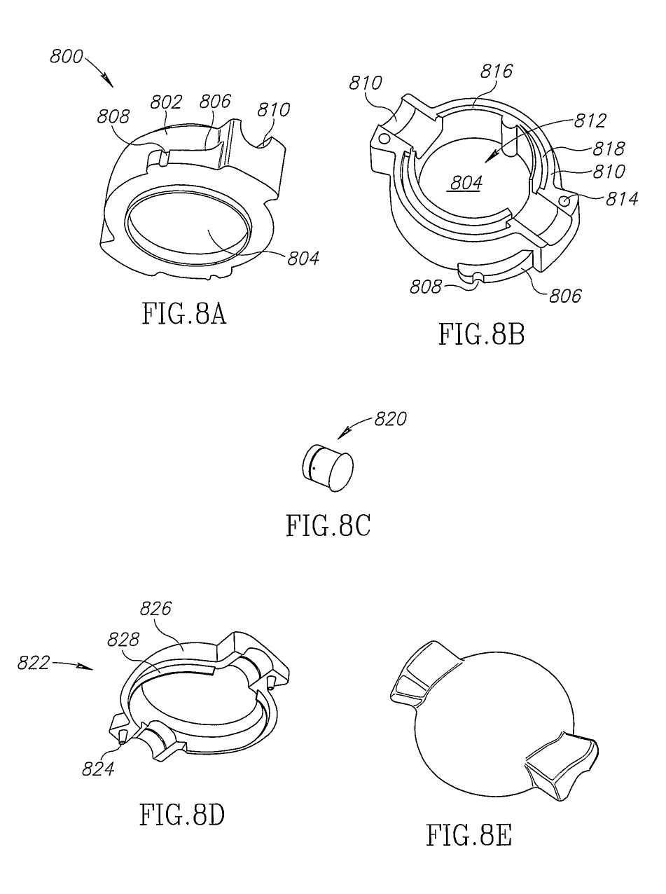

FIGS. 8A-8E show a housing section in accordance with an exemplary embodiment of the invention, for interlocking with element 622 and sticker section 600. In an exemplary embodiment of the invention, he housing section is made of two parts, a base section 800 shown in FIGS. 8A and 8B and a cap section 822 shown in FIGS. 8D and 8E. An optional plug 820 (FIG. 8C) may be used to plug a cable opening in the housing. Optionally, one or more of the sections are attached to each other or to the parts shown in FIG. 6, during manufacture, for example using adhesive.

Referring to FIGS. 8A and 8B, are perspective views from bottom and top respectively of section 800. Section 800 comprises a ring-shaped body 802 with an optional bottom 804. Optionally, a transmitter or sensor element (not shown) is attached to the underside of bottom 804. Alternatively, bottom 804 may be dispensed with or may be apertured. Optionally, an acoustic damping element is provided at bottom 804. The sensors may be, for example, a single element, optionally shaped to have a desired beam-forming behavior or an array. Optionally, the sensor is a transponder. Alternatively, some sensors are elements that are used as transmitters. An optional wing 806 is shown on one side of body 802 (and another one is optionally provided on the hidden side). A notch 808 for engaging beam 630 (FIG. 6), is optionally provided. Wing 806 optionally has a gradually increasing height, to assist in snug engagement of the housing section against the skin of patient 110.

Electronics components (not shown) are optionally provided inside a hollow 812 defined inside body 802. A rim 816 optionally extends from body 802, for engaging cap section 822. One or more, optionally two, cable outlets 810 are defined in body 802. One or more alignment holes 814 which match alignment posts in cap section 822 are optionally provided. Other alignment and/or attachment mechanisms may be provided.

FIGS. 8D and 8E are perspective views from bottom and top respectively of cap section 822. Posts 824 match holes 814. A rim 828 in a cap body 826 optionally matches a groove 818 in body 802. In an exemplary embodiment of the invention, one or more of sections 800, 802, 600 and 620 are marked so that the orientation of the unit as a whole can be aligned to the maternal body axes. Optionally, tongue 614 is used for such orientation.

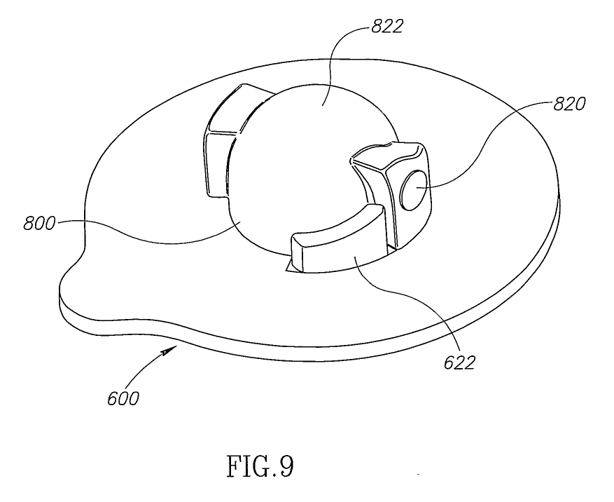

FIG. 9 shows a completely assembled unit, such as can be used for units 508-512.

In some embodiments of the invention, units 508-512 are used as references for internal cervical sensors 150. Considering the geometry of the abdomen, when the mother changes posture, the abdomen may spill to one side and move the sensors. Also, the angle of the surface may change. In an exemplary embodiment of the invention, the inclination sensor is calibrated to have its axes parallel to the longitudinal and the lateral axes of the mother, measuring the roll. As noted above, the housings used are optionally marked to help alignment. The inside of the housing is optionally designed to be suitable to receive the inclination sensor in only one orientation. Optionally, the sensor is calibrated during manufacture to be aligned to the housing in a known way.

In an exemplary embodiment of the invention, after probe placement, the quality of ultrasonic reception is tested. Thereafter, the position of units 508-512 and/or their orientation is marked to system 100. Optionally, the initial measurements are used to calibrate the position and/or orientation, for example based on a known internal geometry. Alternatively or additionally, only changes from an initial positioning are determined.

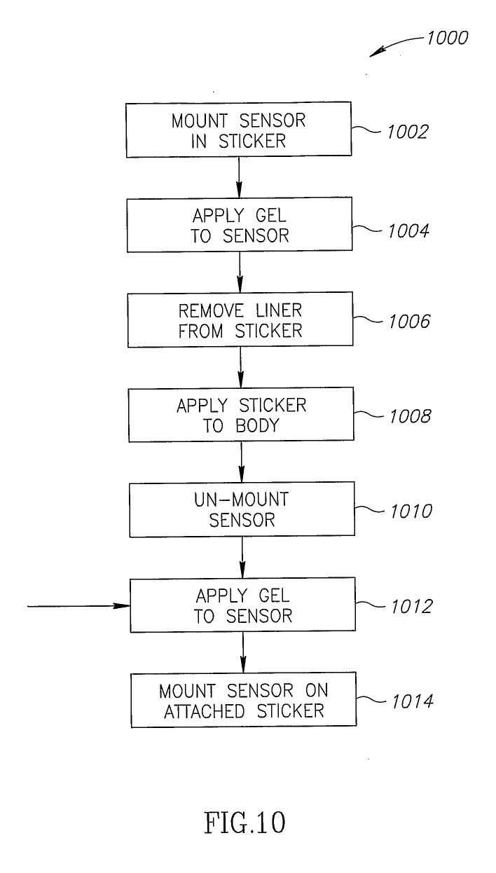

When the mounting system of FIGS. 5-9 is used for ultrasonic transmitters, it is generally accepted to place a layer of acoustic coupling gel (or other coupling material) between the transmitters and the skin of patient 110. Over the course of treating patient 110, one or more of the transmitters may be removed and then replaced. FIG. 10 is a flowchart 1000 of a process of applying gel to a transmitter, in accordance with an exemplary embodiment of the invention.

At 1002, a sensor is mounted in a unit 150, so that the sensor (or a coating thereof) extends through aperture 606.

At 1004, gel is applied to the sensor extending through unit 150.

At 1006, any excess gel is removed by stripping off liner 610 which protects the foam. Thus, in one step, the foam is ready for adhesion and excess gel is removed.

At 1008, the sticker is applied to the skin of patient 110. Optionally, gel is provided in a bladder between the adhesive layer and the body, which bladder is burst by the application, or burst manually (e.g., using a needle or tear line), to spill its gel in a desired location. Optionally, one or more overflow apertures are formed in ring 622 or section 800, to allow excess gel to be extruded away from the sensor/adhesive.

At 1010, the sensor is un-mounted, for example if the patient is being moved, treated, showered or otherwise manipulated in a manner incompatible with the sensors.

At 1012 (which can also be the initial mounting step), gel is applied to the un-mounted sensor. Alternatively, gel is applied directly to the skin through aperture 606.

At 1014, the sensor with gel is snap-mounted onto element 622. Other configurations of component disassembly may be used, for example, the ultrasonic transducer itself may be in the housing (e.g., and be disposable) and the electronics be removable and having contact pads to contact the transducer.

While the above has described using an inclination sensor for detecting change in posture, a same type of sensor may be used instead or in addition to measure maternal parameters, for example, contractions and/or breathing. In an exemplary embodiment of the invention, a halter is provided and the inclination sensor detects contractions. Optionally, the existence of contractions are used to assist in a determination if a change in geometry is an artifact or caused by (a concurrent) contraction. Alternatively or additionally, strength and/or frequency of contraction are used to generate an alert that labor has progressed to a further state. In another embodiment, breathing and, optionally depth of breathing are detected by the inclination sensor. Optionally, the depth of breathing is used as a parameter, like posture, which might affect fetal movement.

It will be appreciated that the above described methods may be varied in many ways. It should also be appreciated that the above described description of methods and apparatus are to be interpreted as including apparatus for carrying out the methods and methods of using the apparatus.

The present invention has been described using non-limiting detailed descriptions of embodiments thereof that are provided by way of example and are not intended to limit the scope of the invention. It should be understood that features and/or steps described with respect to one embodiment may be used with other embodiments and that not all embodiments of the invention have all of the features and/or steps shown in a particular figure or described with respect to one of the embodiments. Variations of embodiments described will occur to persons of the art.

It is noted that some of the above described embodiments may describe the best mode contemplated by the inventors and therefore may include structure, acts or details of structures and acts that may not be essential to the invention and which are described as examples. Structure and acts described herein are replaceable by equivalents which perform the same function, even if the structure or acts are different, as known in the art. Therefore, the scope of the invention is limited only by the elements and limitations as used in the claims. When used in the following claims, the terms “comprise”, “include”, “have” and their conjugates mean “including but not limited to”.

1. A birth monitoring system, comprising:
a monitor including at least one sensor operative to measure a physiological parameter associated with labor;
a posture sensor which generates a signal indicative of posture of a mother; and
circuitry which generates an output signal dependent on both a measurement of said sensor and a posture measurement of said posture sensor.

2. A system according to claim 1, wherein said output signal comprises a signal selectively suppressed responsive to said measured posture.

3. A system according to claim 1, wherein said circuitry is operative to modify said output signal responsive to said posture signal.

4. A system according to claim 1, wherein said circuitry is operative to generate a signal indicative of a change in said physiological parameter responsive to a change in said posture.

5. A system according to claim 1, wherein said physiological parameter comprises a geometry of a cervix.

6. A system according to claim 1, wherein said physiological parameter comprises a fetal head position relative to a birth canal of a mother.

7. A system according to claim 1, wherein said physiological parameter comprises a fetal physiological parameter.

8. A system according to claim 7, wherein said fetal physiological parameter comprises a fetal heart rate.

9. A system according to claim 1, wherein said physiological parameter comprises a maternal physiological parameter.

10. A system according to claim 1, wherein said physiological parameter is changed by a change in posture.

11. A system according to claim 1, wherein a measurement by said sensor is changed by a change in posture.

12. A system according to claim 11, wherein said sensor comprises at least one internal probe and at least one external probe serving as a reference thereto.

13. A system according to claim 1, wherein said output signal is generated if a posture change is not maintained.

14. A system according to claim 1, comprising memory which stores a correspondence between posture and a physiological parameter.

15. A system according to claim 1, wherein said circuitry extracts at least one maternal physiological parameter from said posture sensor.

16. A system according to claim 15, wherein said maternal parameter comprises breathing.

17. A system according to claim 1, wherein said posture sensor comprises an acceleration sensor.

18. A system according to claim 1, wherein said posture sensor is calibrated to be aligned to one or more maternal body axes.

19. A system according to claim 1, wherein said output signal comprises a recommendation to a caregiver regarding posture.

20. A system according to claim 1, wherein said posture sensor is housed together with at least a part of said at least one sensor.

21. A method of generating an output signal indicative of a medical monitoring of a patient in labor, comprising:
measuring at least one postural parameter of the patient;
measuring at least one physiological parameter associated with labor; and
generating a signal responsive to both of said measurements.

22. A method according to claim 21, comprising discarding a physiological measurement responsive to a posture measurement.

23. A method according to claim 21, wherein said generating comprises correcting a physiological measurement based on said posture measurement.

24. A method according to claim 21, wherein said generating comprises tracking a change in at least one physiological parameter responsive to said posture measurement.

25. A method according to claim 21, wherein said generating comprises generating a physiological measurement from said posture measurement.

26. A method according to claim 21, comprising monitoring an effect of posture change on said physiological parameter.

27. A method according to claim 21, comprising monitoring a compliance of a patient with a posture change.

28. A method according to claim 21, comprising determining a side on which the patient is lying.

29. A method according to claim 21, wherein said postural parameter and said physiological parameter are both acquired using a same sensor.

30. A method according to claim 29, comprising determining a change in posture based on a measured change in said at least one physiological parameter.

31. A method according to claim 23, wherein correcting comprises applying a correction value to said physiological parameter.

32. A method according to claim 31, comprising updating said correction value when a posture change is detected.

33. A method according to claim 32, wherein updating comprises updating assuming that said physiological parameter does not change over a period of time of the occurrence of said posture change.

34. A mounting assembly, comprising:
an elastic adhesive ring adapted to adhere to a human skin;
a selectively locking mount attached to said ring; and
a housing adapted to receive a sensor and selectively lockable to said mount, and configured to provide mechanical contact of at least one part thereof to a skin to which the ring adheres.

35. An assembly according to claim 34, wherein said locking is a snap mounting.

36. An assembly according to claim 34, wherein said mount interlocks with at least one aperture in said ring.

37. An assembly according to claim 34, wherein said sensor comprises an ultrasonic transducer adapted to ultrasonically communicate through a center of said ring and wherein said mechanical contact is suitable for ultrasonic transmission therethrough.

38. An assembly according to claim 34, wherein said sensor comprises an inclination sensor.

39. An assembly according to claim 34, comprising circuitry for RF communication.

40. An assembly according to claim 34, comprising a power source in said housing.

41. An assembly according to claim 34, comprising circuitry for digitizing and processing of ultrasound signals.

42. A method of applying ultrasound coupling material to an active element designed for gel-coupled contact with skin, comprising:
mounting the element in an adhesive mount;
applying a coupling material suitable for ultrasonic coupling to the element; and
stripping a covering from said mount, thereby exposing an adhesive layer of said mount and removing excess gel-like material.
